(19) United States
(12) Patent Application Publication
(10) Pub. No.: US 2006/0083786 A1
(43) Pub. Date: Apr. 20, 2006

(54) Taste masking pharmaceutical composition containing levocetirizine

(75) Inventors: Ghanshyam Narayan Chaudhari, Vaidhaihee (IN); Vasant Sitaram Khachane, Nasik (IN); Vaibhav Panditrao Deshmukh, District Nashik (IN); Nitin Babulal Bhamre, Nashik (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/193,542

(22) Filed: Jul. 29, 2005

Related U.S. Application Data
(60) Provisional application No. 60/592,041, filed on Jul. 29, 2004.

(51) Int. Cl. A61K 31/495; A61K 9/26

(52) U.S. Cl. 424/470; 514/255.04

(57) ABSTRACT

A solid oral dosage composition is provided comprising a prophilactically or therapeutically effective amount of an active pharmaceutical ingredient comprising levocetirizine or a pharmaceutically acceptable salt thereof, the solid oral dosage composition having a coating thereon capable of providing taste masking of the levocetirizine or pharmaceutically acceptable salt thereof.

### PRIORITY

This application claims the benefit under 35 U.S.C. §119 to Provisional Application No. 60/592,041, filed Jul. 29, 2004 and entitled “EFFERMELT LEVOCETIRIZINE TABLET”, the contents of which are incorporated by reference herein.

### BACKGROUND OF THE INVENTION

1. Technical Field

The present invention relates generally to a pharmaceutical composition that provides taste masking for levocetirizine or a pharmaceutically acceptable salt thereof.

2. Description of the Related Art

An important therapeutic area in human health is the medicinal treatment of allergic conditions. There has been a significant increase in the number of reported allergic conditions over the last three decades. This is evidenced by the increased levels of antibodies developed in response to environmental allergic factors, e.g., dust mites, pets, and air pollutants, which is specified in, for example, the American Journal Of Respiratory and Critical Care Medicines, 159:125-29 (1999). Related therapies generally involves the use of effervescent tablets, mouth dissolving tablets, oral liquid preparations and the like where the drug is released from the dosage form, absorbed in the blood stream and then reaches the site of action. In its normal state, the body has its own defense mechanism to fight attacking allergens by producing its own antibodies. However, under certain circumstances the body's defense mechanism fails and the patient may then require external medicinal treatment.

An allergy or hypersensitivity is an abnormal reaction to protein substances that occur naturally. If an allergic person is exposed to these substances called allergens, the body's immune system gets ready to fight them. White blood cells (B-lymphocytes) produce an antidote (i.e., antibody) against the allergen. The antibody sticks to the surface of the allergy cells. At this point, the body is ready to fight back the next time it is exposed to the allergen. This process is called sensitization.

After this change, there is an allergic reaction every time the body is exposed to the allergen. The allergen sticks to the antibodies on the surface of the allergy cells. This coupling causes the granula (little stores in the allergy cells) to release a histamine, which causes the symptoms of allergy. Depending on the size of the exposure to the allergen, and where on the body it happens, there will be an allerg-icy reaction in the form of, for example, hay fever, asthma or nettle rash.

The histamine dilates the blood vessels, causing the mucous membrane (tissue lining of the nose and airways) to swell due to the liquid leaking and stimulates the glands in the nose and the respiratory passages to produce mucus (i.e., phlegm). Substances that make the musculature of the respiratory passages contract are released along with the histamine, it then becomes difficult to breath and an asthma attack may follow.

Also, allergens are the microscopic protein substances that are common and provoke allergic people to produce antidotes (antibodies). Some of the most common allergy provoking substances are for example, pollen from weeds, grass, flowers, trees, mould, fungus, house dust mites, fur from cats and dogs, and certain medicines.

Individuals sensitive to allergic reactions have very sensitive mucous membranes, which can be irritated by many different substances including smoke, pollution, cooking smells, perfume and strong odors. Children who are passively smoking are more at risk of developing allergic reactions. The predisposition to the hypersensitivity is hereditary. For example, if one or both parents or close family members suffer from hypersensitivity, this condition will continue into the next generation.

Allergies are frequently treated by drugs called antihistamines. Examples of such antihistamines are Claritin and Allegra, but antihistamines have been in use since as early as the 1930s and they continue to be an effective way to deal with the problems of allergies. One effect antihistamines have is the histamine mediated release of inflammatory mediators by leukocytes. Other antihistamine effects result in the neutralization of histamine, preventing it from binding and activating immune related cells in the area. An allergic reaction is an immune response that should not be occurring because the substance that triggers it should not be dangerous to us. Allowing our immune system to run its course against allergens means living with annoying and potentially dangerous symptoms. The use of antihistamines allows us to live more safely and comfortably by counteracting the body's immunological mistakes.

Certain drugs therapies that may be effective in the treatment of allergies, referred to as first generation antihistamines such as hydroxizine, are effective, but possess the major disadvantage that they cause sedation. The initial second generation antihistamines, terfanadine and astemizole, were effective non-sedating medications but had drug interactions that caused cardiac problems. Later second generation antihistamines, such as loratadine and citirizine, have been found to be effective in the treatment of allergic rhinitis and the latter to be effective in the treatment of chronic idiopathic urticaria.

Cetirizine is an orally active, long-acting, histamine H₁ receptor antagonist. Antihistamines, such as cetirizine, block the effect of histamines that are released by allergic reactions in the body. This mitigates the ability of histamine to promote allergy symptoms. The non-sedating character of cetirizine is an important breakthrough in allergy treatment because new generation allergy drugs diminish the commonly experienced, sedative effect and allow patient to enjoy an improved quality of life.

Cetirizine has an asymmetric center in the molecule, and thus may exist as optical isomers (enantiomers). Levocetirizine is the R-enantiomer of cetirizine. Levocetirizine, like cetirizine, has a potential anti-inflammatory effect in the treatment of allergic rhinitis with asthma. Levocetirizine is believed to have a two fold higher affinity for human H₁ receptors than cetirizine. Levocetirizine is also believed to be rapidly and extensively absorbed. Levocetirizine has been shown to be free from side effects on the central nervous system. See, e.g., Journal of Allergy and Clinical Immunology, 111:3, pp. 623-627 (2003).

U.S. Patent Application Publication No. 2004/0132743 (“the '743 application”) discloses the amorphous form of levocetirizine dihydrochloride. The '743 application further discloses pharmaceutical compositions that include a prophylactically or therapeutically effective amount of the amorphous form of levocetirizine dihydrochloride that is substantially free of its crystalline form.

It would be desirable to provide a solid pharmaceutical composition containing levocetirizine levocetirizine or a pharmaceutically acceptable salt thereof which has a coating capable of providing taste masking of the levocetirizine or a pharmaceutically acceptable salt thereof.

### SUMMARY OF THE INVENTION

In accordance with one embodiment of the present invention, a solid oral dosage composition is provided comprising a prophilactically or therapeutically effective amount of an active pharmaceutical ingredient comprising levocetirizine or a pharmaceutically acceptable salt thereof, the solid oral dosage composition having a coating thereon capable of providing taste masking of the levocetirizine or pharmaceutically acceptable salt thereof.

In accordance with a second embodiment of the present invention, a pharmaceutical composition is provided comprising a core region comprising a therapeutically effective amount of levocetirizine or a pharmaceutically acceptable salt thereof and a taste-masking film coating layer over at least a portion of the core.

In accordance with a third embodiment of the present invention, a tablet is provided comprising a core region comprising a therapeutically effective amount of levocetirizine or a pharmaceutically acceptable salt thereof and a taste-masking film coating layer over at least a portion of the core.

In accordance with a fourth embodiment of the present invention, a method for preparing an effermelt tablet is provided comprising (a) slugging levocetirizine or a pharmaceutically acceptable salt thereof with other excipients; de-slugging the product of step (a) to form granules and (c) tabletting the granules into a tablet dosage form.

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Unless stated to the contrary, any use of the words such as “including”, “containing”, “comprising”, “having” and the like, means “including without limitation” and shall not be construed to limit any general statement that it the specific or similar items or matters immediately following it. Except where the context indicates to the contrary, all exemplary values are intended to be fictitious, unrelated to actual entities and are used for purposes of illustration only. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention is defined by the appended claims.

The expression “solid oral dosage composition” as used herein shall be understood to mean all solid oral dosage forms including powders, tablets, dispersible granules, capsules, caplets, sachets and the like.

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” means the amount of a compound that, when administered for treating or preventing a disease, is sufficient to effect such treatment or prevention for the disease. The “therapeutically effective amount” will vary depending on the compound, the disease and its severity and the age, weight, etc., of the patient to be treated.

The term “delivering” as used herein means providing a therapeutically effective amount of an active ingredient to a particular location within a host means causing a therapeutically effective blood concentration of the active ingredient at the particular location.

The term “pharmaceutically acceptable” as used herein means that which is useful in preparing a pharmaceutical composition that is generally non-toxic and is not biologically undesirable and includes that which is acceptable for veterinary use and/or human pharmaceutical use.

The term “subject” or “a patient” or “a host” as used herein refers to mammalian animals, preferably human.

The term “pharmaceutical composition” is intended to encompass a product comprising the active ingredient(s), pharmaceutically acceptable excipients that make up the carrier, as well as any product which results, directly or indirectly, from combination, complexation or aggregation of any two or more of the ingredients, or from dissociation of one or more of the ingredients. Accordingly, the pharmaceutical compositions of the present invention can encompass any composition made by admixing the substantially crystalline form of levocetirizine, optionally one or more additional active ingredient(s) and one or more pharmaceutically acceptable excipients.

The term “excipients” as used herein means a component of a pharmaceutical product that is not an active ingredient such as, for example, fillers, diluents, carriers and on the like. The excipients that are useful in preparing a pharmaceutical composition are preferably generally safe, non-toxic and neither biologically nor otherwise undesirable, and are acceptable for veterinary use as well as human pharmaceutical use.

As used herein, the term “adsorbant” is intended to mean an agent whose surface area is larger to the irregularity and roughness of the surface. The active chemical entity gets bound on this surface by forming Van der Waals interactions, and hydrogen bonding forces of attraction (forms physical bond) and thus the drug surface is completely covered by the adsorbing material, in turn helps in masking the bitter taste of the pharmaceutically active entity.

As used herein, the term “sweetener” is intended to mean an agent who imparts the artificial sweetening to the formulation to help in masking the unacceptable taste for the dosage form. Useful sweeteners include, but are not limited to, sodium saccharine, dextrose, sucrose, aspartame, magnasweet and the like and mixtures thereof. In the present invention aspartame is the preferred sweetening agent.

As used herein, the term “disintegrating agent” is intended to mean an agent which in contact with water swells and fragments the solid dosage form. Useful disintegrating agents include, but are not limited to, croscarmellose sodium, crospovidone, and the like and mixtures thereof. In the present invention, crospovidone may be used as a disintegrating agent, which swells in the presence of water and helps in fragmenting the tablet dosage form. Additional exemplary disintegrants include, by way of example and without limitation, starches, e.g., sodium starch glycollate, corn starch, potato starch, pre-gelatinixed and modified starched thereof; clays, e.g., bentonite, microcrystalline cellulose (e.g. Avicel™), carsium (e.g. Amberlite™), alginates, sodium starch glycolate, gums, e.g., agar, guar, locust bean, karaya, pectin, tragacanth, combinations thereof and other such materials known to those of ordinary skill in the art.

As used herein, the term “effervescing agent” is intended to mean such materials known to those of ordinary skill in the art. Here citric acid is used in the dry mix intragranularly and sodium bicarbonate (dried) is used extra granularly. These ingredients in the presence of water undergo a chemical reaction to evolve carbon dioxide by forming effervescence.

In the present embodiment the flavorings agents can be used intragranularly and extragranulerly. The flavorings agents are chemical moieties such as esters and aldehydes which imparts the flavoring effect to the dosage forms.

As used herein the term “antioxidant” is intended to mean an agent which inhibits oxidation and is thus used to prevent the deterioration of preparations by the oxidative process. Such compounds include, by way of example and without limitation, ascorbic acid, ascorbic palmitate, Vitamin E, butylated hydroxyanisole, butylated hydroxytoluene, hypophosphorous acid, monothioglycerol, propyl gallate, sodium ascorbate, sodium bisulfite, sodium formaldehyde sulfoxylate, sodium metalbisultfite and other such materials known to those of ordinary skill in the art.

As used herein, the term “buffering agent” is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

As used herein, the term “binders” is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include, but are not limited to, starch, poly(ethylene glycol), guar gum, polysaccharide, bentonites, sugars, invert sugars, poloxamers (PLURONIC™ F68, PLURONIC™ f127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

As used herein, the term “diluent” or “filler” is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

As used herein, the term “glidant” is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

As used herein, the terms “lubricant” or “lubricating agent” is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

As used herein, the term “wetting agent” is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogel emulsifying wax, sorbitan esters, polyoxyethylene alkyl ethers (e.g., macrogel ethers such as cetomacrogol 1000), polyoxyethylene castor oil derivatives, polyoxethylene sorbitan fatty acid esters, (e.g., TWEEN™s), polyethylene glycols, polyoxyethylene stearates colloidal silicon dioxide, phosphates, sodium dodecylsulfate, carboxmethylcellulose calcium, carboxmethylcellulose sodium, methylcellulose, hydroxethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymet of the alkyl aryl polyether alcohol type, also known as superinone or triton) is another useful wetting agent, combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### DETAILED DESCRIPTION OF THE PREFERRED EMBODIMENTS

The present invention is directed to a solid oral dosage composition comprising a prophilactically or therapeutically effective amount of an active pharmaceutical ingredient comprising levocetirizine or a pharmaceutically acceptable salt thereof, the solid oral dosage composition having a coating thereon capable of providing taste masking of the levocetirizine or pharmaceutically acceptable salt thereof. Any form of levocetirizine or pharmaceutically acceptable salt thereof can be used herein. See, for example, U.S. Pat. No. 6,319,927 and U.S. Patent Application Publication Nos. 2004/0132743 and 2005/0038039, the contents of which are incorporated by reference herein.

The pharmaceutical compositions of the present invention may contain one or more additional active pharmaceutical ingredients (APIs). Useful additional APIs include, but are not limited to, anti-histaminic agents and pharmaceutically acceptable salts thereof and the like and combinations thereof. Suitable antihistaminic agents include, but are not limited to, ethanolamines, e.g., imenhydrinate, diphenhydramine and the like; alkylamines, e.g., chlorpheniramine, dexchlorpheniramine and the like; phenothiazines, e.g., promethazines and the like; piperazines, e.g., hydroxyzines and the like; piperadines, e.g., cyproheptadine azatadine and the like and combinations thereof.

In one embodiment, the pharmaceutical compositions can be a fast melting solid dosage form, e.g., an effermelt tablet, designed to rapidly disintegrate in the oral cavity, thus allowing patients to overcome the problems of swallowing cumbersome dosage forms, which discourages many from taking their medication. In this manner, the fast melting dosage form combines the benefits of liquid formulations with those of a solid oral dosage form resulting in improved patient compliance by providing patients with a more convenient means of taking their medication. The solid dosage form can include at least a core region containing the levocetirizine or pharmaceutically acceptable salt thereof.

To provide a fast melting dosage form, the solid oral pharmaceutical composition of this invention will include a taste-masking film coating layer. The coating layer may be an adsorbent material covering at least a portion of the solid dosage form containing the foregoing active pharmaceutical ingredients which advantageously provides the taste masking effect to overcome the bitter tasting active pharmaceutical ingredients. The adsorbent has an irregular surface area and thus provides a larger surface area. Accordingly, the adsorbent material can surround the drug particles by forming a physical bond, by Van der Waals interactions, and hydrogen bonding force of attraction, so the bitter tasting drug is not capable of coming into contact with the taste buds while consuming the effermelt dosage form. The structure form is capable of releasing the active pharmaceutical ingredient at the site of action. Suitable adsorbents includes, e.g., pharmaceutically acceptable adsorbents. In one embodiment of the present invention, aluminum magnesium silicate is used as an adsorbent. Because of its silicate content, it acts like an adsorbent. Also, the adsorbent properties is also dependent upon the content of silica in the compound. Other effective adsorbing agents to mask the taste of the bitterly tasting active ingredients, include, but are not limited to, zeolite, activated granular carbon, silica gel, active aluminum, clays and the like and mixtures thereof.

In another embodiment of the present invention, a solid pharmaceutical formulation includes, for example, taste-masked microcapsules, having (a) a core containing at least levocetirizine or pharmaceutically acceptable salt thereof, and (b) a polymeric coating capable of providing taste-masked characteristics, the coating containing a mixture of at least two water insoluble polymers, at least one of which is capable of forming a film. Both the core and the polymeric coating may further contain one or more pharmaceutically acceptable excipients, e.g., diluents, fillers and other pharmaceutical additives which may effect the rate of release of active agent(s) from the microcapsule.

Representative film forming, water insoluble polymers for use in the taste-masking film coating layer of the composition of the present invention include, but are not limited to, ethylcellulose, e.g., Ethocel™ available from Dow Chemical Corp., aqueous polymeric dispersions such as Aquacoat™ (an about 30% w/w aqueous dispersion containing ethyl cellulose, sodium lauryl sulfate, cetyl alcohol and hydrogen peroxide with a pH of about 4.0-7.0) available from FMC, and Surelease™ (a plasticized 25% w/w aqueous dispersion containing ethyl cellulose, ammonium hydroxide, medium chain triglycerides & oleic acid with a pH of about 9.5-11.5) available from Colorcon, polyvinyl acetate, cellulose acetate butyrate, and copolymers of polymethacrylic acid available from Rohm Pharma GmbH under the tradename Eudragit™ (e.g., Eudragit L30D-55, Eudragit L100-55, Eudragit RS30D and Eudragit RL30D). Most preferably, the film forming, water insoluble polymer of the present invention is ethylcellulose.

The second water insoluble polymer of the for use in the taste-masking film coating layer advantageously dissolves under acidic conditions of the stomach (e.g., gastric juices), e.g., at a pH level of about 5 or below. Preferably, the polymer is a methacrylic or polymethacrylic acid copolymer. A preferred polymethacrylic acid copolymer is available from Rohm Pharma GmbH under the tradename Eudragit™ (e.g., Eudragit L30D-55, Eudragit L100-55, Eudragit RS30D and Eudragit RL30D). Most preferably, the pH dependent copolymer of the present invention is Eudragit EPO (Rohm Pharma). The pH dependent polymer is present as discrete particles in the coating.

According to the present invention, once a patient places the composition in the mouth, the coating layer substantially maintains its integrity during the brief transit period in the mouth. The coating layer remains intact because the pH dependent polymer will only dissolve once it is exposed to acidic conditions of the stomach, i.e., at a pH of about 5 or below, which is much more acidic than the pH of the mouth. Once the composition enters the acidic environment of the stomach, dissolution occurs and the medication is then available for absorption by the body.

By employing a polymeric coating composition containing a mixture of at least two water insoluble polymers, dispersible in water, aqueous coating techniques can advantageously be used. Aqueous-based coating systems are relatively more safe and make regulatory compliance relatively easy compared to non-aqueous based coating systems. A number of polymeric coatings that can provide an elastic microcapsule and will not release the active agent in the mouth when administered are contemplated by the present invention.

The coating layer of the present invention can be applied to the cores described hereinabove by conventional techniques. For example, spray techniques such as top spray, bottom spray and tangential spray techniques using, for example, a fluidized bed coater. For example, air (which may be heated) passes through a bed of the active ingredient granules to fluidize them, and the aqueous solution of the two water insoluble polymers is sprayed onto the fluidized bed and thereby coats the granules. The air passing through the bed dries the coated granules, so that a dry coated granule is obtained. The coated granules can then be used in combination with various excipients, flavors, and colors to make a taste-masking film coated solid oral dosage composition of the present invention.

The dried coating as applied generally varies from about 5 percent by weight to about 30 percent by weight of the total dry weight of the coated composition. Preferably, the coating varies from about 10 percent by weight to about 20 percent by weight of the total dry weight of the coated composition. The exact proportions of coating to the core desired for individual cases can be determined by routine experimentation. The amount of coating may be varied in light of the intended application and desired bulk of the products.

Generally, the solid pharmaceutical compositions of the present invention can be prepared by any method known in the art. For example, a core can be prepared by extrusion-spheronization (where drug(s) and other additives are granulated by addition of a binder solution. The wet mass is passed through, for example, an extruder equipped with a certain size screen. The extrudates can then be spheronized in a marumerizer. The resulting pellets are dried and sieved for further applications); high-shear granulation (where drug(s) and other additives are dry-mixed and then the mixture is wetted by addition of a binder solution in a high shear-granulator/mixer. The granules can be kneaded after wetting by the combined actions of mixing and milling. The resulting granules or pellets are dried and sieved for further applications); and the like. If desired, one or more pharmaceutically active ingredients or pharmaceutically acceptable excipients can be present in the core region. Examples of pharmaceutically acceptable ingredients or excipients include, but are not limited to, binders, disintegrant agents, fillers, surfactants, stabilizers, lubricants, glidants, antioxidant and the like and mixtures thereof. The active ingredient of the invention may also be administered via fast dispersing or fast dissolving dosage forms or in the form of a high energy-dispersion or as coated particles.

In yet another embodiment of the present invention, the core of the composition can contain various pharmaceutical additives in combination with the API requiring taste-masking. Examples of such additives include, but are not limited to, antioxidants, buffering agents, sweetening agents, binders, diluents, fillers, glidants, lubricating agents, disintegrants, wetting agents and the like and mixtures thereof. Each of the foregoing additives, when used, is used at a functionally effective amount to impart the desired properties to the pharmaceutical formulations herein.

In one embodiment of the present invention, the content of the core in the composition of the present invention generally varies from about 70 percent by weight to about 95 percent by weight, based on the total weight of the composition. Preferably, the core varies from about 80 percent by weight to about 90 percent by weight of the total coated composition. The shape and size of the core is not limited and may range from truly spherical to irregular and non-uniform. Preferably, the core is finished to granules having a size ranging from about 150 to about 500 microns.

The more preferred oral solid preparation is a tablet. A tablet may be prepared by direct compression, wet granulation, or molding, of the substantially crystalline form of levocetirizine dihydrochloride with a carrier and other excipients in a manner known to those skilled in the art. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made on a suitable machine, a mixture of the powdered compound moistened with an inert liquid diluent, are suitable in the case of oral solid dosage forms (e.g., powders, capsules, and tablets). The levocetirizine or pharmaceutically acceptable salt thereof may be formulated into a typical disintegrating tablet in any release form, e.g., controlled release, extended release, or immediate release dosage forms.

Preferably, each tablet contains from about 2.5 mg to about 10 mg of levocetirizine or pharmaceutically acceptable salt thereof, and each cachet or capsule contains from about 2.5 mg to about 10 mg of levocetirizine or pharmaceutically acceptable salt thereof. Most preferably, the tablet contains about 5 mg of levocetirizine or pharmaceutically acceptable salt thereof for oral administration. The prophylactic or therapeutic dose of levocetirizine or pharmaceutically acceptable salt thereof can vary widely depending upon a variety of factors including, for example, the body weight, general health, sex, diet, time and route of administration, rates of absorption and excretion, combination with other drugs, the severity of the particular condition being treated, etc. The dose and perhaps the dose frequency will also vary according to the age, body weight and response of the individual patient. In general, the total daily dose range for levocetirizine or pharmaceutically acceptable salt thereof is from about 2.5 mg to about 10 mg. Preferably, a daily dose range should be about 5 mg to about 10 mg in single or divided doses. Most preferably, the dose range is about 5 mg per day. It is known that children and elderly patients, as well as those with impaired renal or hepatic function, should receive low doses, at least initially.

The pharmaceutical compositions of the present invention can contain one or more pharmaceutically acceptable excipients in accordance with known and established practice. The amount of the additional pharmaceutically acceptable excipients generally varies from about 10% to about 90% by weight, based on the total weight of the total composition. The pharmaceutically acceptable excipients include, but are not limited to, fillers, glidants, lubricants, bulking agents, disintegrating agents and the like that are typically used in the art for oral solid dosage forms.

Suitable fillers for use herein may be, for example, inert fillers, either water soluble or water insoluble and selected from those typically used in the pharmaceutical art for oral solid dosage forms. Examples include, but are not limited to, lactose (monohydrate/dihydrate), starch dried, aspartame, crospovidone, mannitol (Perlitol SD 200), sorbitol, sodium chloride and the like and mixtures thereof. The amount of filler varies widely and will ordinarily range from about 1% to about 90% by weight, based on the total weight of the composition.

Suitable glidants for use herein can be any glidant typically used in the pharmaceutical art for oral solid dosage forms. Examples include, but are not limited to, colloidal silicon dioxide, talc and the like and mixtures thereof. The amount of glidant varies widely and will ordinarily range from about 0.1% to about 5.0% by weight, based on the total weight of the composition.

Suitable lubricants for use herein can be any lubricant typically used in the pharmaceutical art for oral solid dosage forms. Example include, but are not limited to, stearate salts such as calcium stearate, magnesium stearate, and zinc stearate, stearic acid, talc, hydrogenated vegetable oil, vegetable oil derivatives, silica, silicones, high molecular weight polyalkylene glycols and saturated fatty acids and the like and mixtures thereof. The amount of lubricant varies widely and will ordinarily range from about 0.1% to about 5.0% by weight, based on the total weight of the composition.

Examples of a bulking agent include lactose monohydrate, cornstarch (dried), mannitol and the like and mixtures thereof. The amount of bulking agent varies widely and will ordinarily range from about 4% to about 15% by weight, based on the total weight of the composition.

A suitable disintegrating agent includes crospovidone which is available as a white, free flowing compressible powder. Crospovidone is a cross-linked N-vinyl-2-pyrrolidone homopolymer. It is completely insoluble in water, acids, alkalis and all organic solvents. Crospovidone may also help in enhancing the dissolution of pharmaceutically active substance and in turn enhances the bioavailability for the same.

Another excipient that may be used in the present invention is submicroscopic fumed silica with a particle size of about 15 nm. It is a light, loose, bluish-white colored, odorless, tasteless, gritless amorphous powder. As colloidal silicon dioxide has a small particle size and a large surface area give it desirable flow characteristics, and so used as a glidant to improve the flow ability of the dried granules.

The pharmaceutical composition of the present invention can further contain agents that generate effervescence e.g. anhydrous citric acid and sodium bicarbonate (dried), which in contact with water evolves carbon dioxide and produces effervescence.

Formulations for oral use of the pharmaceutical compositions of the present invention can be provided as effervescent mouth dissolving tablets wherein the pharmacologically active ingredients are mixed with the inert excipients to form the effervescent mouth dissolving tablet pharmaceutical composition of the present invention according to procedures known in the art.

In another embodiment of the present invention, oral dosage forms of the pharmaceutical compositions herein can be obtained by (a) slugging the active pharmaceutical ingredient along with diluents; (b) deslugging the slugs obtained in step (b); (c) lubricating the product of step (b) with a lubricant; and (d) tableting the mixture.

The following example is provided to enable one skilled in the art to practice the invention and is merely illustrative of the invention. The examples should not be read as limiting the scope of the claims.

### EXAMPLE 1

### Preparation of an Effermelt Anti-Histaminic Tablet

Suitable ingredients for use in this example are set forth below in Table 1. The product can be made by a slugging process and incorporating the active drug as intragranular component.

TABLE 1

| | | |
|---|---|---|
| Ingredients/Components | Qty (mgs) | % w/w |

I. MANUFACTURING OF LEVOCETIRIZINE
DIHYDROCHLORIDE GRANULES

| | | |
|---|---|---|
| Levocetirizine Dihydrochloride (crystalline) | 5.10 | 2.372 |
| AluminiumMagnesium Silicate(Veegum HV) | 12.00 | 5.581 |
| Lactose(Directly Compressible) | 46.85 | 21.791 |
| Starch (Dried) | 30.00 | 13.954 |
| Lake of Ponceau 4 R | 0.30 | 0.140 |
| Aspartame | 20.00 | 9.302 |
| Crospovodone | 5.50 | 2.558 |
| Anhydrous Citric Acid | 18.50 | 8.605 |
| Sodium Chloride | 2.00 | 0.932 |
| Colloidal Silicon Dioxide | 1.00 | 0.465 |
| Magnesium Stearate | 0.90 | 0.419 |

II. LUBRICATION

| | | |
|---|---|---|
| Starch(Dried) | 12.45 | 5.791 |
| Lake of Ponceau 4 R | 0.10 | 0.047 |
| Sodium Bicarbonate (Dried) | 27.75 | 12.907 |
| Mannitol(Perlitol SD 200) | 20.00 | 9.302 |
| Crospovidone | 5.25 | 2.442 |
| Firmenich Tutti Fruity (colorcon) | 5.00 | 2.326 |
| Firmenich Powder Flavor Mint (colorcon) | 0.40 | 0.186 |
| Colloidal Silicon Dioxide | 1.00 | 0.465 |
| Magnesium Stearate | 0.90 | 0.418 |

Relative Humidity during manufacturing and packaging of the tablet should be maintained between about 25 to about 30% and room temperature between about 21 to about 25° C.

Preparation of the Tablet

Step I: Manufacturing of Levocetirizine Dihydrochloride Granules

Levocetirizine Dihydrochloride and aluminium magnesium silicate (Veegum HV) were sifted using a mechanical sifter through a 40# sieve and mixed thoroughly in an octagonal blender for two to three minutes. 3 min. Lactose (directly compressible) was then added to the mixture and mixed. Starch dried and lake Ponceau 4 R were sifted through 40# sieve and collected in a separate polybag lined container. Aspartame, crospovidone, anhydrous citric acid, sodium chloride and colloidal silicon dioxide were sifted through 40# sieve and collect in a separate polybag and magnesium stearate was added.

Levocetirizine dihydrochloride, aluminium magnesium silicate (Veegum HV), lactose (directly compressible), starch (dried), Lake Ponceau 4R, aspartame, crospovidone, anhydrous citric acid, sodium chloride, colloidal silicon dioxide were mixed for 15 minutes an magnesium stearate was added to the mixture and mixed for an additional 3 minutes. The blend was unloaded in polybag lined drums.

Step II: Slugging

The blend from step I was slugged using a 16 mm Round biplane puncher with a 16/27 station rotary compression machine. The slugs were then sifted through an Oscillating Granulator using an 8#, a 12# and a 18# sieve or multimill the slugs through 6 mm SS screen (750 rpm), 4 mm SS screen (750, 1500 rpm), a 2 mm SS screen (1500 rpm) and then passed through a 18# sieve with about 3 kg fine powder being kept for mixing with the lubricants.

Sodium bicarbonate was sifted using a mechanical sifter through a 60# sieve. Next, the sifted sodium bicarbonate was dried in a Tray Dryer at 100° C. for 1 hour and unloaded in a dehumidified area. The weight was then measured and any loss on drying was compensated by adding an additional quantity of sodium bicarbonate (previously dried).

Step III: Lubrication

Starch and Lake Ponceau 4R were sifted using mechanical sifter through a 0# sieve and collected in a polybag lined container. Sodium bicarbonate, mannitol (Perlitol SD 200), crospovidone , firmenich powder tuttifruity (colorcon) and firmenich powder flavor mint (colorcon), colloidal silicon dioxide were sifted through a 40# sieve and collected in a polybag. Next, sodium bicarbonate, mannitol (Perlitol SD 200), crospovidone, firmenich powder tuttifruity (colorcon), firmenich powder flavor mint (colorcon), and colloidal silicon dioxide were added to the polybag to form a lubrication premix.

The granules from step II were then loaded in an octagonal blender and mixed for 5 minutes. Next, the lubrication premix was added in the blender and mixed for 15 minutes. Magnesium stearate was then added and mixed for an additional 3 minutes. The lubricated granules were then put in polybag lined drums.

Step IV: Compression

The lubricated granules from step III were compressed into tablets using 8.4 mm flat-faced beveled edge punches with breakline on upper punch and lower punch plain at 16/27 station rotary compression machine observing the specifications. The tablets were compressed with an average compression weight of 215 grams and a hardness of 2-4 kg/cm2.

### EXAMPLE 2

The tablets (5 mg) prepared in Example 1 were then subjected to a dissolution study by storing the tablets at a temperature of 25° C.±2° C. and a relative humidity (RH) of 60%±5% for 24 months. The results of the study are set forth in Table 2.

TABLE 2

| | Initial | Analysis After | | | |
|---|---|---|---|---|---|
| Tests | analysis | 3 Months | 6 Months | 9 Months | 12 Months |
| Appearance | Complies | Complies | Complies | Complies | Complies |
| Avg. Wt. of Tablets in mg | 215.4 | 215.5 | 214.7 | 216.6 | 216.4 |
| Diameter avg. in mm | 8.43 | 8.40 | 8.42 | 8.44 | 8.38 |
| (8.30 mm to 8.50 mm) | | | | | |
| Thickness avg. in mm | 2.95 | 2.99 | 2.97 | 2.98 | 2.99 |
| (2.70 mm to 3.10 mm) | | | | | |
| Hardness in kg/cm² | 3.0 | 4.0 | 3.0 | 5.0 | 4.0 |
| (NLT 2.0 kg/cm²) | | | | | |
| Friability in % | 0.21% | 0.25% | 0.20% | 0.22% | 0.26% |
| w/w (NMT 1.0%) | | | | | |
| Dissolution (Lt: NLT 70% of the | 95.6% | 94.3% | 93.1% | 92.8% | 93.5% |
| labelled amount of drug is dissolved | | | | | |
| in 30 minutes) | | | | | |
| Disintegration Time (NMT 5 min using | 47 sec | 55 sec | 48 sec | 1 Mins 10 sec | 1 Mins 15 sec |
| water at 20 to 30° C.) | | | | | |
| Assay of Levocetirizine Dihydrochloride | 102.3% | 102.2% | 101.8% | 101.5% | 101.3% |
| (90.0% to 110.0% of labeled amount) | | | | | |

The following observations were made for changes in the physical and chemical characteristics of the tablets up to 12 months.
1. No change in the physical characteristics of the tablet was observed.
2. There was no indication of interaction of packing material (aluminum strip using foil of 0.03 mm thickness) with the tablets.
3. The chemical characteristics of the product was found to be within specified limits.

### EXAMPLE 3

The tablets (5 mg) prepared in Example 1 were then subjected to a dissolution study by storing the tablets at a temperature of 40° C.±2° C. and a relative humidity (RH) of 75%±5% for 6 months. The results of the study are set forth in Table 3.

TABLE 3

| | Initial | Analysis After | | | |
|---|---|---|---|---|---|
| Tests | analysis | 1 Month | 2 Months | 3 Months | 6 Months |
| Date of Analysis | 12/03/04 | 15/04/04 | 13/05/04 | 11/06/04 | 14/09/04 |
| Appearance | Complies | Complies | Complies | Complies | Complies |
| Avg. Wt. of Tablets in mg | 215.4 | 215.6 | 215.7 | 216.3 | 216.6 |
| Diameter avg. in mm | 8.43 | 8.42 | 8.44 | 8.39 | 8.41 |
| (8.30 mm to 8.50 mm) | | | | | |
| Thickness avg. in mm | 2.95 | 2.98 | 3.02 | 3.04 | 3.01 |
| (2.70 mm to 3.10 mm) | | | | | |
| Hardness in kg/cm² | 3.0 | 3.0 | 4.0 | 4.0 | 5.0 |
| (NLT 2.0 kg/cm²) | | | | | |
| Friability in % w/w | 0.21% | 0.23% | 0.21% | 0.23% | 0.24% |
| (NMT 1.0%) | | | | | |
| Dissolution | 95.6% | 95.4% | 93.7% | 92.5% | 91.9% |
| (Lt: NLT 70% of the | | | | | |
| labelled amount of drug | | | | | |
| is dissolved in 30 minutes) | | | | | |
| Disintegration Time | 47 sec | 57 sec | 59 sec | 56 sec | 1 Mins 23 sec |
| (NMT 5 min using water | | | | | |
| at 20 to 30° C.) | | | | | |
| Assay of Levocetirizine | 102.3% | 102.1% | 101.6% | 101.7% | 101.4% |
| Dihydrochloride | | | | | |
| (90.0% to 110.0% of | | | | | |
| labeled amount) | | | | | |

The following observations were made for changes in the physical and chemical characteristics of the tablets.
1. No change in the physical characteristics of the tablet was observed.
2. There was no indication of interaction of packing material (aluminum strip using foil of 0.03 mm thickness) with the tablets.
3. The chemical characteristics of the product was found to be within specified limits.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

What is claimed is:

1. A solid oral dosage composition comprising a prophilactically or therapeutically effective amount of an active pharmaceutical ingredient comprising levocetirizine or a pharmaceutically acceptable salt thereof, the solid oral dosage composition having a coating thereon capable of providing taste masking of the levocetirizine or pharmaceutically acceptable salt thereof.

2. The solid oral dosage composition of claim 1, wherein the coating comprises an adsorbent material.

3. The solid oral dosage composition of claim 2, wherein the adsorbent material is selected from the group consisting of aluminum magnesium silicate, zeolite, activated granular carbon, silica gel, active aluminum, clay and mixtures thereof.

4. The solid oral dosage composition of claim 1, wherein the coating comprises (a) a film-forming, water-insoluble polymer; and, (b) a pH dependent, water-insoluble polymer which dissolves at a pH level of about 5 or below.

5. The solid oral dosage composition of claim 4, wherein the film-forming, water-insoluble polymer is selected from the group consisting of ethylcellulose, polyvinyl acetate, cellulose acetate butyrate, methacrylic acid copolymers and mixtures thereof.

6. The solid oral dosage composition of claim 4, wherein the pH dependent, water-insoluble polymer is a copolymer.

7. The solid oral dosage composition of claim 6, wherein the copolymer is a methacrylic acid copolymer or a polymethacrylic acid copolymer.

8. The solid oral dosage composition of claim 4, wherein the film-forming, water-insoluble polymer is ethylcellulose and the pH dependent, water-insoluble polymer is a methacrylic acid copolymer.

9. The solid oral dosage composition of claim 4, wherein the film-forming, water insoluble polymer is present in an amount from about 25 wt. % to about 75 wt. % and the pH dependent, water-insoluble polymer is present in an amount from about 30 wt. % to about 70 wt. %, based on the total weight of the coating layer.

10. The solid oral dosage composition of claim 4, wherein the film forming, water insoluble polymer is present in an amount from about 40 wt. % to about 60 wt. % and the pH dependent, water-insoluble polymer is present in an amount from about 35 wt. % to about 55 wt. %, based on the total weight of the coating layer.

11. The solid oral dosage composition of claim 4, wherein the pH dependent, water-insoluble polymer is present as discrete particles distributed homogeneously throughout the coating layer.

12. The solid oral dosage composition of claim 1, which is in the form of a tablet.

13. The solid oral dosage composition of claim 2, which is in the form of a tablet.

14. The solid oral dosage composition of claim 4, which is in the form of a tablet.

15. The solid oral dosage composition of claim 1, further comprising one or more pharmaceutically acceptable excipients.

16. A taste-masking solid pharmaceutical composition comprising:
(a) a core region comprising an active pharmaceutical ingredient comprising levocetirizine or a pharmaceutically acceptable salt thereof and at least one pharmaceutically acceptable excipient; and
(b) a taste-masking film coating layer over the core.

17. The taste-masking solid pharmaceutical composition of claim 16, wherein the amount of the active pharmaceutical ingredient in the core is from about 5 wt. % to about 35 wt. %, based on the total weight of the core.

18. The taste-masking solid pharmaceutical composition of claim 16, wherein the core is present in an amount of about 70 wt. % to about 95 wt. % and the coating is present in an amount of about 5 wt. % to about wt. 30 wt. %, based on the weight of the composition.

19. The taste-masking solid pharmaceutical composition of claim 16, wherein the core is present in an amount of about 80 wt. % to about 90 wt. % and the coating is present in an amount of about 10 wt. % to about 20 wt. %, based on the weight of the composition.

20. The taste-masking solid pharmaceutical composition of claim 16, wherein the coating comprises an adsorbent material.

21. The taste-masking solid pharmaceutical composition of claim 20, wherein the adsorbent material is selected from the group consisting of aluminum magnesium silicate, zeolite, activated granular carbon, silica gel, active aluminum, clay and mixtures thereof.

22. The taste-masking solid pharmaceutical composition of claim 16, wherein the coating comprises (a) a film-forming, water-insoluble polymer; and, (b) a pH dependent, water-insoluble polymer which dissolves at a pH level of about 5 or below.

23. The taste-masking solid pharmaceutical composition of claim 22, wherein the film-forming, water insoluble polymer is present in an amount from about 25 wt. % to about 75 wt. % and the pH dependent, water-insoluble polymer is present in an amount from about 30 wt. % to about 70 wt. %, based on the total weight of the coating layer.

24. The taste-masking solid pharmaceutical composition of claim 22, wherein the film-forming, water-insoluble polymer is selected from the group consisting of ethylcellulose, polyvinyl acetate, cellulose acetate butyrate, methacrylic acid copolymers and mixtures thereof.

25. The taste-masking solid pharmaceutical composition of claim 22, wherein the pH dependent, water-insoluble polymer is a copolymer.

26. The taste-masking solid pharmaceutical composition of claim 22, wherein the copolymer is a methacrylic acid copolymer or a polymethacrylic acid copolymer.

27. The taste-masking solid pharmaceutical composition of claim 22, wherein the film-forming, water-insoluble polymer is ethylcellulose and the pH dependent, water-insoluble polymer is a methacrylic acid copolymer.

28. A process for the preparation of a solid oral dosage composition of claim 1, which comprises
(a) slugging the levocetirizine or a pharmaceutically acceptable salt thereof with an adsorbent material and one or more pharmaceutically acceptable excipients to provide slugs;
(b) deslugging the slugs and granulating the slugs;
(c) lubricating the granules; and
(d) tableting the granules in the desired shape and size.
